(19) United States
(12) Patent Application Publication
(10) Pub. No.: US 2023/0075606 A1
(43) Pub. Date: Mar. 9, 2023

(54) METHOD FOR PRODUCING AND DISCHARGING ULTRAPURE HYDROGEN PEROXIDE GAS INTO THE AMBIENT AIR, RELATED EQUIPMENT, SPECIAL POLYMER NANOCOMPOSITE, AND USES

(71) Applicant: NANOATIVA NANOTECNOLOGIA ATIVAL LTDA, Florianopolis (BR)

(72) Inventor: Valeria VIDAL DE OLIVEIRA, Florianopolis (BR)

(21) Appl. No.: 17/916,809

(22) PCT Filed: Mar. 9, 2021
(86) PCT No.: PCT/BR2021/050099

§ 371 Date: Oct. 4, 2022

(30) Foreign Application Priority Data
Apr. 5, 2020 (BR) BR102020006855 5

(51) Int. Cl. A61L 2/20; A61L 9/04; B01J 21/06; B01J 19/12

(52) U.S. Cl. CPC A61L 2/208; A61L 9/04; B01J 21/06; B01J 19/123; A61L 2209/211; A61L 2202/11

(57) ABSTRACT

Method for producing and discharging ultrapure hydrogen peroxide gas GPHU into the ambient air, said gas being substantially free of hygroscopic substances and substantially free of metals, primarily for use in bio-oxidative treatments via the blood stream by inhalation, for use in humans and animals. Said method comprises ultrapure hydrogen peroxide gas, alkaline nanostructured nanomaterial metal catalyst, special polymer nanocomposite material NPE and UV light. The ultrapure hydrogen peroxide gas is discharged into the ambient air naturally by the surface of the NPE. Equipment for producing and discharging ultrapure hydrogen peroxide gas into the ambient air is also disclosed.

### FIELD OF THE INVENTION

The following descriptive report for invention refers to method for synthesising and releasing in the environment Ultrapure Hydrogen Peroxide Gas GPHU. The said method is formed by Ultrapure Hydrogen Peroxide Gas, metal catalyst made of an alkaline nanostructured nanomaterial, a Special Polymeric Nanocomposite NPE material, and UV light. The NPE material is formed by metal catalyst made of an alkaline nanostructured nanomaterial and by synthetic polymer. In said method, the releasing of GPHU is carried out naturally by the surface of the photoactivated NPE material. The invention also refers to Ultrapure Hydrogen Peroxide, to the NPE material and to the apparatus for implementing this method. The apparatus is specially use for the production and release of GPHU for use in bioxidative therapies via the bloodstream by inhalation, applied to humans and animals, formed by NPE, UVA light and electric cable to connect UVA light to electric energy. This equipment can also be used for the purpose of biodisinfection of air and surfaces in empty environments or in inhabited environments.

### BACKGROUND OF THE INVENTION

In the state of the art when the compound hydrogen peroxide H₂O₂ dissolved in an aqueous solution, the said solution is called oxygenated water. In the other hand, When the Hydrogen peroxide compound is released into the gaseous environment for uses, it can be termed in several ways, depending mainly on its purity of its production pathway.

When the Hydrogen peroxide compound is derived from the vaporization of aqueous solutions containing dissolved hydrogen peroxide, it is called Hydrogen Peroxide Vapor VHP. When it comes from the ‘drying’ of Hydrogen Peroxide Vapor, it is called Hydrogen Peroxide Gas GHP. When it comes from catalytic reactions, it is termed according with the type of catalytic reaction in Dry Hydrogen Peroxide DHP when it is obtained via electrocatalysis, and in pure hydrogen peroxide gas PHPG or pure hydrogen peroxide gas nonhidrate when it is obtained via photocatalysis. There may be other denominations. Both DHP and PHPG are produced by catalytic reactions in a gaseous state, an anhydrous/dry pathway, unlike VHF and GHP that come from aqueous solutions containing dissolved hydrogen peroxide, a wet pathway.

The VHF is not a gas, it is formed by liquid particles containing dissolved hydrogen peroxide. GHP despite being considered a gas, is formed by a hydrated form of hydrogen peroxide compound H₂O₂. H₂O. Already DHF and PHPG are pure gases, that is, they are free of water molecules. The DHP and PHPG are similar, distinguished only by the catalytic pathway that gave rise to it, electrocatalysis and photocatalysis, respectively.

The production of pure hydrogen peroxide gases can be achieved from any process described in the technique where catalytic reactions involving the oxidation of water in the gaseous or liquid state occur, or another compound that can provide hydrogen ions that can be separated by permeation, with simultaneous reduction of oxygen, such as occurs in the catalytic processes of photocatalysis and electrocatalysis, and there may be others, without being limited to them.

However, despite providing the production of pure hydrogen peroxide gases; catalytic processes do not provide their uses and effects in the environment, because uses and effects are only possible if it; the pure hydrogen peroxide gas is in the environment; and in catalytic processes concomitantly with the production of pure hydrogen peroxide gas, there is also the production of a plasma containing highly reactive radicals, such as reactive species of oxygen ERO and hydroxyl radicals, located very close to the activated catalytic surface. And the catalytic plasma, oxidizes newly generated pure hydrogen peroxide gas to hydroxyl radicals. In addition Consumption of the newly generated pure hydrogen peroxide gas, can also occur on the activated catalytic surface: Pure hydrogen peroxide gas can act as electron acceptor in the conduction band of activated catalyst, suffering reduction in place of oxygen, by being more electronegative than oxygen, reducing to hydroxyl radicals and oxygen. Pure hydrogen peroxide gas can also be consumed by its direct reaction with gaseous pollutants, or its reaction, of pure hydrogen peroxide gas, with the products resulting from its own oxidation/reduction.

To promote the uses and effects of pure hydrogen peroxide gas in the environment, it is necessary that there is a morphology or structure near the activated catalytic cell, which removes the newly formed pure hydrogen peroxide gas from near it, before it, the pure hydrogen peroxide gas, is consumed by the system itself. For this, the contact time of said gas with the activated catalytic surface must be less than 1 second.

In the state of the art Patent U.S. Pat. No. 8,168,122B2, granted on May 1, 2012, and the worldwide applications related to it, and Patent U.S. Pat. No. 9,808,013B2, granted on Nov. 7, 2017, and the worldwide applications related to it, refer to PHPG; and POT WO2020077263A1, published on Apr. 16, 2020, and related worldwide applications, refers to DHP. In these references, the concentration in the steady state of PHPG/DHP in the treatment environment is up to 0.1 ppm, and the use of GPHU/DHP is for microbial control, disinfection and remediation of air and surfaces in environments. The presence of PHPG/DHP in the environment achieved due to the use of a forced airflow passing through a mesh containing activated particulate metal oxide catalysts, deposited superficially on the said mesh, and after, the airflow returns to the environment, carrying with it the DHP/PHPG and other compounds derived from catalytic reactions, launching them into the environment. The one speed rate is between 5 nm/s to 10,000 nm/s, Patent U.S. Pat. No. 9,808,013B2 and POT WO2020077263A1 still bring in common the presence of hygroscopic additive covering the surface of said catalysts.

In the state of the Technique DHP/PHPG is considered: a) pure because it is free of water molecules, because it is obtained by a dry route; b) substantially plasma-free, because together with the release of DHP/PHPG to the environment there is no release of plasma species, because they, the plasma species, have a very short life time, less than 1 millisecond, and are not released to the environment; and c) substantially free of ozone gas, because together with the release of DHP/PHPG to the environment there is the release of ozone gas in concentration below its perception limit of 0,015 ppm.

Other uses of DHP/PHPG may include arthropod control, including insects and arachnids, for GPHU concentrations above 0.1 ppm in the environment, according to U.S. Pat. No. 9,808,013B2, granted on Nov. 7, 2017, and related worldwide applications; (e) to improve the health of the respiratory system, increase resistance to infection and increase hypothionat ion in the lungs of mammals, humans and animals, according to Patent AU2014308887B2, granted on 26 Jul. 2018, and related worldwide applications; and for poultry production, specifically the disinfection of egg surfaces, according to POT WO2018129537A2, published on 12 Jul. 2018, and related worldwide applications.

### SUMMARY OF THE INVENTION

In the present invention is presented a new pure hydrogen peroxide gas, obtained via photocatalysis, called Ultrapure Hydrogen Peroxide Gas GPHU, GPHU is also free of water molecules, plasma-free and substantially free of ozone gas, it is also substantially free of hygroscopic substances and substantially metal-free. Ultrapure quality allows concentration in the average steady state in the GPHU environment of up to 5 ppm, and also allows it to be used as a therapeutic agent in bioxidative therapies via the bloodstream by inhalation. Ultrapure quality is achieved because the invention makes use of metal catalyst made of an alkaline nanostructured nanomaterial, with the following main characteristics: it is a nanostructured nanomaterial with dimensionality 1D and/or 2D and/or 3D; it is absent from hygroscopic substances covering its surface; has affinity with water due to the presence of alkalis in its structure; it is superficially coated with organic additive; is photoactivated preferably by UVA light; is a structural element of a composite material.

In the present invention the releasing of GPHU in the environment enabling its uses and effects is carried out naturally by the surface of the Special Polymeric Nanocomposite NPE material, at an average speed rate between 2,2×10¹¹ nm/s at 4,4×10¹¹ nm/s. The natural release of the inventive method is achieved due to the special configuration of the NPE material, which encompasses the following main characteristics: opaque, porous to gases and with roughness on at least one of the faces.

### BRIEF DESCRIPTION OF FIGURES

FIG. 1 shows the diphratogram with the crystalline structure of the NPE material marked with the Symbol (f-n−1) and the Symbol (f-n−2), and the diphrate with the crystalline structure of the metallic catalyst made of an alkaline nanostructured nanomaterial with the Symbol (f-n−3). The metal catalyst made of an alkaline nanostructured nanomaterial is sodium titanate, and thermoformable synthetic polymer is polyethylene (f-n−1) and polypropylene (f-n−2). Analysis obtained by the analytical technique of X-ray Diffraction, in Philips X′Pert Difratometer, with copper tube, 1,54 angstrom radiation, and 0,02°/s scan, Peak identification was done using Philips software, based on JCPDS.

FIG. 2 shows the morphology of the metallic catalyst made of an alkaline nanostructured nanomaterial with 2D dimensionality, of sodium titanate with nanowalls formed, well distributed in the NPE material. The image was obtained by the analytical technique of Electron Microscopy of Scanning MEV-FEG. The equipment data were 5.0 KV×100,000 (A) and ×50,000 (B), WD 6.1 mm, scale ruler at 100 nm.

FIG. 3 shows the Electronic Reflectance Spectrum of the metal catalyst made of alkaline nanostructured nanomaterial, sodium titanate. The analysis performed by the electronic absorption spectroscopy technique with specular reflectance measurements in the UV-Vis region. The symbol (A) shows the peak absorption of UV light energy by the said catalyst at 372 nm, contained in the UVA light energy range.

FIG. 4 shows the plant of the device for production and release in the gaseous environment of GPHU for use. From left to right and top to bottom are the top view, front view, bottom view, front section showing the inner view, and perspective.

FIG. 5 with the symbol (A) shows the image of papaya fruit packed in a cold chamber for 32 days. The symbol “V” shows the sample of papaya packed for 32 days in cold chamber without the photoactivated NPE material. The symbol “2” shows the papaya sample packed for 32 days in a cold chamber containing plates of NPE material photoactivated by UVA light, connected every 15 min per hour.

FIG. 5 with the symbol (B) shows the plates of UVA light-photoactivated NPE material, used in a cold chamber for the analysis of the conservation of fruits and vegetables foods.

### DETAILED DESCRIPTION

This patent application refers to method for production and release to the environment for uses of Ultrapure Hydrogen Peroxide Gas GPHU. The said method is formed by Ultrapure Hydrogen Peroxide Gas, metal catalyst made of an alkaline nanostructured nanomaterial, Special Polymeric Nanocomposite NPE material, and UV light. Additionally, the invention refers to apparatus for the production and release of GPHU to the gaseous environment, for use in bioxidative therapies via the bloodstream by inhalation, applied to humans and animals, and for use in the biodisinfection of air and surfaces of empty environments or inhabited environments. The device is formed by NPE material, UVA light, and power cable to connect UVA light to the power grid.

The production mechanism of GPHU is via photocatalysis, whose mechanism begins when a photon of UV light reaches the surface of the metal catalyst of alkaline nanostructured nanomaterial, contained in the NPE material, with energy equal to or greater than the band gap energy of said catalyst. In the conduction band of said catalyst are generated spare electrons and, and in the valence band of said catalyst are generated positive gaps It, according to Equation 1, causing reactions by electron transfer, mainly between the said catalyst and the molecular oxygen O2 and the water present in the environment. Water acts as an electron donor, or a hydroxyl ion, in the valence band of said catalyst, releasing H according to Equation 2; Equation 3 and Equation 4; and The O₂ it acts as an electron acceptor in the conduction band, forming superoxide anions in the system, which can be involved in other reactions, generating other oxidizing compounds, according to Equation 5 and Equation 6. The end of the reaction chain occurs with the generation GPHU/H₂O_{2(g)} free of water molecules, according to Equation 7.

catalyst+hv→e⁻+h⁺ Equation 1

h⁺+RX_{ad}→RX_{ad}⁺ Equation 2

h⁺+H₂O_{ad}→OH·_{ad}+H⁺ Equation 3

h⁺+OH_{ads}⁻→OH·_{ads} Equation 4

e⁻+O₂→O₂⁻ Equation 5

O₂⁻+H⁺→HO₂· Equation 6

H⁺+O₂⁻+HO₂·⁻→H₂O_{2(g)}+O₂ Equation 7

The GPHU as soon as it is produced is released naturally by the surface of the photoactivated NPE material.

Photoactivated NPE material can be used in open systems for production and release to the GPHU, i.e. the photoactivated NPE material can be used directly in the environment without being involved or inside a enclosure.

Photoactivated NPE material can be used in closed systems for production and release to the GPHU, i.e., the photoactivated NPE material may be involved or inside a enclosure. In this case, so that GPHU released by the surface of the photoactivated NPE material inside the casing is not consumed inside it, in the casing, it is necessary a forced airflow between the casing and the photoactivated NPE material or forced airflow between the UV light and NPE material, or even a forced airflow between casing and photoactivated NPE material and a forced airflow between the light UV and NPE material. The airflow must have a speed rate that ensures that the GPHU is less than one second inside the enclosure, thus allowing it, GPHU released into the enclosure, to be “dragged” by the airflow out of the casing and released to the environment before it is consumed by the other system products or reduced by the catalyst. In another alternative, for example, the photoactivated NPE material may be in an open system and on it be released a forced airflow. In these cases the airflow is only a release adjunct, because the GPHU release is initially performed naturally by the surface of the photoactivated NPE material. The fact is that the use of photoactivated NPE material for the production and release to the GPHU can occur in several ways, in addition to those already exposed, characterized by containing the photoactivated NPE material.

Preferably, the photoactivated NPE material is used in open systems for production and releasing GPHU. In this case, the release of GPHU occurs naturally through the surface of the NPE Photoactivated material, without the need to use complementary equipment for the launch.

GPHU is a gas with a molar mass of 34,0147 grams per mol (g/mol), formed by the compound pure hydrogen peroxide in the gaseous state H₂O_{2(g)}. The gas molecules are always moving at high speed, and the higher the temperature, the faster they, the molecules of the gases, move. Low density molecules move faster than heavy molecules at the same temperature. At room temperature, a technician on the subject knows that oxygen gas moves at an average speed of 1600 kilometers per hour (km/h), equivalent to 4,4×10¹¹ nanometers per second (nm/s), and that the molar mass of oxygen gas O₂ is 32 g/mol. Knowing also that the molar mass of hydrogen gas H₂ is 2 g/mol, then the rate of release of GPHU to the environment naturally by the surface of the photoactivated NPE material is between 2,2×10¹¹ nm/s to 4,4×10¹¹ nm/s. When in the environment it, the GPHU, can be used and its effects checked.

When it is in the environment, the average concentration in a stationary state of GPHU is between 0,001 parts per million to 5 parts per million. The analysis to determine the average concentration in stationary state of GPHU in the aqueous environment was performed by the permanganometry technique. The releasing of GPHU in the aqueous environment followed the following methodology: in a beaker 100 ml of ultrapure water was added, and then the beaker was closed with a round NPE plate. The beaker was then hermetically sealed between the contact surface of the beaker edge with the NPE. 30 W UVA light was used to photoactivate the NPE. The activated NPE area was 30 cm², One sample was photoactivated for 1 hour, another sample for 2 hours, another sample by 3 h and another sample for 19 hours. All analyses done in triplicate. The result of permanganometry analysis showed average steady state concentration of hydrogen peroxide in water was 5,225±0,53 ppm. In a gaseous environment, the analysis was carried out in a closed environment of 1 cubic meter, using the inventive equipment for the production and release of Hydrogen Peroxide Gas in the environment. The technical data of the analysis were: CAS hydrogen peroxide: 7722-84-1; Reference methodology MA-138 OSHA 1019; analytical technique: Visible absorption spectrophotometry; cassette sampler with two fiberglass membranes impregnated with titanium oxysulfate solution; sampling flow flow 1 to 2 L/min, sample flow of 2 L/min; volume of 5 maximum sampling 240 Liters, sample volume 120 Liters; quantification limit of 5 μg. The minimum concentration of hydrogen peroxide identified by the technique is <0.1 ppm. The steady state concentration of GPHU in a gaseous environment was 4.97 ppm. Thus, by the analyses in water and air, it was possible to establish the average steady state concentration of the GPHU in the ambient of 5 parts per million.

Average concentrations of GPHU above 5 parts per million in a closed environment become unstable: above 5 parts per million the concentration of ozone gas in the environment considerably consumes the concentration of GPHU, according to Equation 8, and it, the concentration of GPHU, reduces in the environment. The working range of ambient air relative humidity is between 1% and 99%, preferably between 40% and 80%.

O₃+H₂O₂→H₂O+2O₂ Equation 8

Together with the releasing of GPHU to the environment naturally by the surface of the fotoactivated NPE material, there may be, likewise, the releasing of ozone gas to the environment, a byproduct of catalytic reactions. GPHU is substantially free of ozone gas. Substantially free of ozone gas means that the concentration of ozone gas in ambient air is below 0.015 ppm. The analysis of the concentration of ozone gas released in the environment, simultaneously to the launch of GPHU; carried out in a closed environment of 1 cubic meter. Technical data from the analysis were: CAS ozone: 10028-15-6; Reference methodology MA-091 OSHA ID 214; analytical technique of ion chromatography; Cassette sampler with 2 glass fiber filters with porosity of 1 mm; impregnated with Sodium Nitrite; sampling flow rate: 0,25 to 1,5 L/min; sampling volume of 90 L; Quantification limit: 0,8 μg. The result was less than 0,005 ppm, which is the minimum value of quantification of ozone gas by the technique. Thus; GPHU is substantially ozone-free. Based on the EPA, through the CRF (Code of Federal Regulations) Tile 21 the maximum exposure of 0.05 ppm of ozone gas established as a standard of indoor air quality.

Together with the release of GPU to the environment naturally by the surface of the photoactivated NPE material, alkalis may be released. Alkali traces may be present in the polymeric matrix of the NPE material, appearing as a byproduct of the synthesis of a metallic catalyst made from alkali nanostructured nanomaterial, originating from alkali metals or alkaline earth metals. Alkalis can react with moisture in the ambient air to form hydroxides. Alkali hydroxides are hygroscopic and hydrophilic alkaline substances. The alkali detection limit in ambient air is <0,1 mg/m³. GPHU is substantially alkali-free/hygroscopic substances. Substantially free of hygroscopic substances means that the concentration of alkalis in ambient air is below the alkali detection limit in ambient air; which is 0.1 mg/ms. The determination of sodium (Na) concentration was performed in a closed environment of 1 cubic meter. The technical data of the analysis were: Sodium CAS: CAS 7440-23-5; reference methodology MA-035 NIOSH 7303; analytical technique of inductively coupled plasma optical emission spectrometry; sampler: cassette with 0,8 μg cellulose ester filter; sampling flow rate: 1 to 4 L/min; Sampling volume: 30 to 960 Liters, sampled 239; 4 Liters; Quantification limit: 3 μg. The result was lower than the detection limit of the inductively coupled plasma optical emission spectrometry analytical technique of <0.1 mg/m³. Thus, GPHU is substantially free of hygroscopic substances.

Together with the release of GPHU to the environment naturally by the surface of the photoactivated NPE material, there may be the release of metal. Metal traces may be present in the polymeric matrix of the NF E material, appearing as a byproduct of the synthesis of the metal catalyst that is made of alkaline nanostructured nanomaterial from metallic oxides. The detection limit of metals in ambient air is <0.1 mg/m³, GPHU is substantially metal-free. Substantially metal-free means that the concentration of metal in ambient air is below 0; 1 mg/m³. For the analysis of the determination of titanium (Ti) concentration, it performed in a closed environment of 1 cubic meter. Technical analysis data were Titanium CAS: 7440-32-6; reference methodology MA-035 NIOSH 7303; analytical technique inductively coupled plasma optical emission spectrometry; sampler: cassette with 0,8 μg cellulose ester filter; sampling flow rate: 1 to 4 L/min, sampled 3.99 L/min; sampling volume: 30 to 960 Liters, sampled 239; 4 Liters; Quantification limit: 0,4 μg. The result was lower than the detection limit of the inductively coupled plasma optical emission spectrometry analytical technique of <0,1 mg/m³. Thus, the GPHU is substantially metal-free, where the concentration of metal particles in ambient air, together with the release of GPHU, is below 0.1 mg m³.

GPHU is free of water molecules, as can be seen in Equation 7. However, when the GPHU is releasing in the aqueous environment, when it exposed to water in it the GPHU dissolves forming oxygenated water from GPHU. The oxygenated water from GPHU can be use in bioxidative therapies, applied to humans and animals, or as antiseptic, among other applications, without limiting themselves to these.

GPHU is plasma-free, A technician on the subject knows that plasma species have a lifetime of milliseconds and there are only very close to the catalytic surface, without being able to release to the environment.

In nanotechnology any material that has at least one nanoscale external dimension, in the length range between 1 nm and 100 nm, is called nanomaterial. Nanomaterials are classified according to their format in 0D, 1 D, 2D and 3D dimensionality.

Nanomaterials of zero Dimension 0D, have all dimensions measured within the nanoscale, no dimension is greater than 100 nm, this class includes the formats of nanoparticles how uniform nanoparticle matrices or quantum dots, arrangements of nanoparticles or duster, core-shell nanoparticles, hollow cubes, nanospheres, nanolenses, among others, without limiting themselves to these.

Unidimensional nanomaterials 10, have two dimensions in nanoscale and one dimension above 100 nm. This class includes the formats of nanotubes, nanorods, nanowires, nanophytes, sintered hierarchical nanostructures, among others, without limiting themselves to these.

Bidimensional nanomaterials 20, have one nanoscale dimension and two dimensions above 100 nm. This class displays plate-like formats and includes graphene, nanofilms, nanolayers, nanocoatings, junctions (continuous islands), branched structures, nanoprisms, nanoplates, nanosheets, nanowalls, nanodisks, among others, without limiting themselves to these.

Three-dimensional nanomaterials 3D, have large specific surface area relative to their mass counterparts, and none nanoscale dimensions. This class includes polycrystals and includes the shapes of nanoflowers, nanocones, nanoballs with dendritic structures, nanopilao, nanocoils, among others, without limiting themselves to these.

Nanostructured nanomaterials are the composition of interrelated constituent parts in which one or more of these parts is a nanoscale region. A region defined by a boundary that represents a discontinuity in the properties. All definitions related to nanotechnology used in this document follow ISO/TS80004.

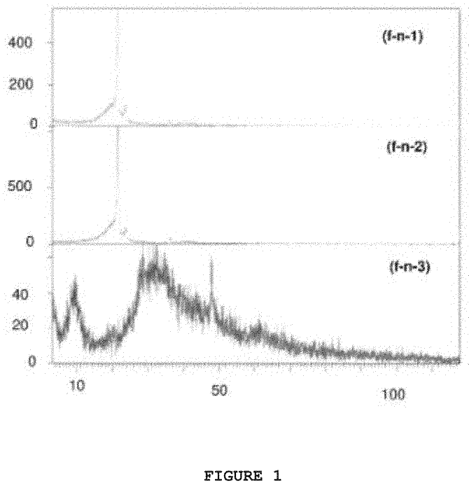

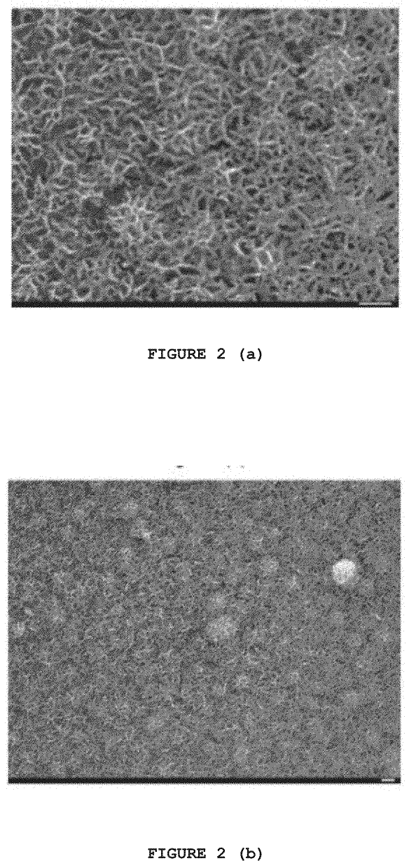

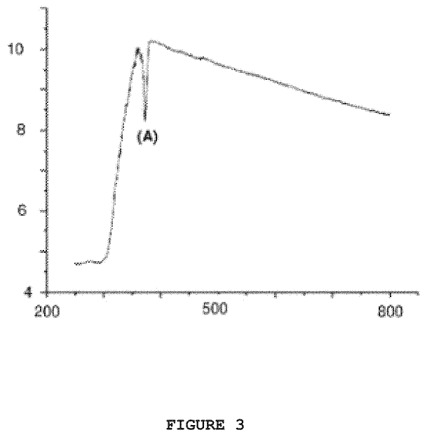

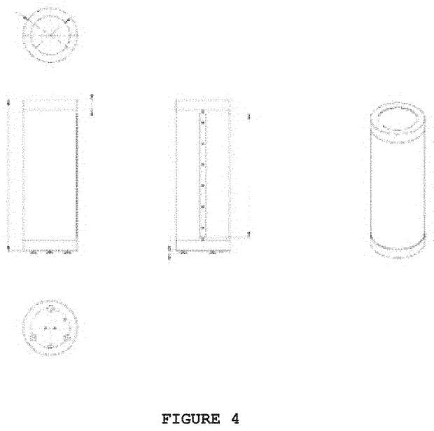

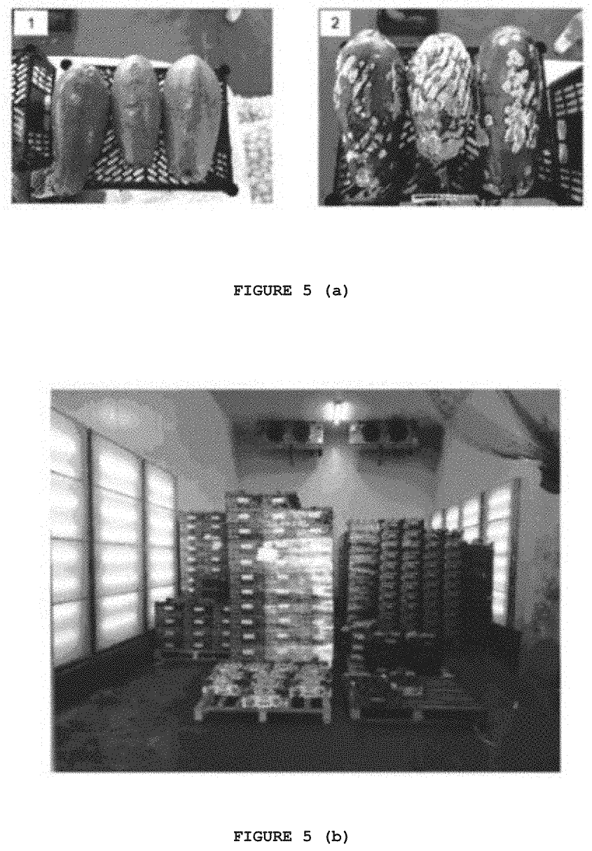

The Special Polymeric Nanocomposite NPE material, is a nanostructured material formed by metal catalyst made of an alkaline nanostructured nanomaterial and by synthetic polymer, where the catalytic property of NPE material is attributed to the presence of the said catalyst in nanoscale, as shown in FIG. 1. The NPE material may also contain untied traces of metallic oxide and alkalis, derived as a byproduct of the synthesis of the metallic catalyst made of an alkaline nanostructured nanomaterial. Traces of metallic oxides and alkali traces mean that there may be very little presence of metal oxides and alkalis in the NPE material, as shown in FIG. 1.

The metal catalyst made of alkaline nanostructured nanomaterial can may present dimensionality 10, or 2D or 3D, or one or more combinations between them. Preferably, the metal catalyst made of alkaline nanostructured nanomaterial presents 2D dimensionality.

The metal catalyst made of alkaline nanostructured nanomaterial is formed by metallic oxide and alkalis in its structure, as shown in FIG. 1.

Metallic oxide is chosen from TiO₂ anatase, or ZnO, or CLIO, or WO₃, or Al₂O₃, or Nb₂O₅, or In₂O₃, or Fe₂O₃, or SnO₂, or SrTiO₃, or Nb₂O₅/MoO₃, or ZnS, or CdS, or one or more combinations between them. Preferably, metallic oxide is TiO₂ anatase. The metallic oxide is nanoparticulate in shape, with an average particle diameter between 1 nanometer and 100 nanometers, preferably with an average particle diameter between 1 nanometer and 30 nanometers. Metallic oxide is nanoparticulate, so metallic oxide has 0D dimensionality. Metallic oxide can may have metallic co-catalyst deposited on its surface, chosen from Ag, Au, or Pt, or Al, or Ru, or Co, or Cr, or Cu, or Rh, or Nb, or Mg, or Si, or Sn, or Pd, or Ti, or Ir, or Zn, or Ge, or Mo, or Fe, or Ni, or W, or Ta, or one or more combinations between them. Preferably the metallic co-catalyst is chosen from Ag, or Au, or Al, or one or more combinations between them.

Alkalis can be alkaline metals or can be alkaline earth metals, or be the combination of them. Alkaline metal is chosen from Li, or Na, or K, or Rb, or Cs, or to be one or more combinations between them. The alkaline earth metals is chosen from Be, Mg, Ca, Sr, Ba, Ra, or from one or more combinations between them. Preferably, alkalis is chosen from Li, or Na, or Mg, or from one or more combinations between them.

Preferably, the alkaline nanostructured nanomaterial metal catalyst is chosen from sodium titanate with 2D dimensionality with nanowalls format, or lithium titanate with 2D dimensionality with nanowalls format, or magnesium titanate with 2D dimensionality with nanowalls format, or be one or more combinations among them.

The metal catalyst of alkaline nanostructured nanomaterial may present metallic co-catalyst deposited on its surface, chosen from Ag, Au, or Pt, or Al, or Ru, or Co, or Cr, or Cu, or Rh, or Nb, or Mg, or Si, or Sn, or Pd, or Ti, or Ir, or Zn, or Ge, or Mo, or Fe, Ni, or W, or Ta, or one or more combinations between them. Preferably, the metallic co-catalyst is chosen from Ag, or Au, or Al, or from one or more combinations between them.

The metal catalyst made of alkaline nanostructured nanomaterial can be produced by any known method that provides to add the crystalline structure of metal oxides other elements such as methods in solid state, xerothermic method, sol-gel, polyol, and hydrothermal, without limiting themselves to these. Preferably, the metal catalyst made of na alkaline nanostructured nanomaterial is produced by the hydrothermal method, as shown in Example 1.

The metal catalyst made of alkaline nanostructured nanomaterial presents surface covered with organic additive for the production of NPE material. The organic additive acts as a conciliator of the immiscible phases between the inorganic nature of the catalyst, with the organic nature of the NPE polymeric matrix, promoting the good distribution of the catalyst in the polymeric matrix of the NPE. In addition, the organic additive has the function of decreasing the cohesive force between the catalysts themselves, also contributing to their distribution in the polymeric matrix of the NPE.

The organic additive may be organic substances or additives, of animal, vegetable or mineral origin, with oily properties or with physical/chemical characteristics compatible with oily properties such as hydrocarbons, fats and esters, preferably chosen between vegetable oil, mineral oil, or fatty material, or the combination between them.

The organic additive can also be a tensoactive agent. The tensoactive agent are substances or additives, with affinity properties for oils, fats and surfaces of solutions with solids, liquids or gases, but also by water, and may belong to both media. The tensoactive agent is with load, and can be cationic, anionic or amphoteric, or the mixture of amphoteric with anionic, or the mixture of amphoteric with cationic, Preferably, the tensoactive agent is CTAB cetyltrimethylammonium bromide.

The organic additive may also be compatibilizing substances or additives of the copolymers type, preferably the graftized copolymer (PP-G-MA), without limiting it.

Preferably the organic additive are organic substances or additives, of animal, plant or mineral origin, with oily or physical/chemical properties compatible with oily properties, such as hydrocarbons, fats and esters, chosen preferably between vegetable oil, mineral oil, or fatty material, or combinations among them. More preferably the organic additive is vegetable oil.

Metal catalyst made of an alkaline nanostructured nanomaterial is well distributed in the polymer matrix of NPE, with absence of agglomeration.

The NPE material is formed by metal catalyst made of alkaline nanostructured nanomaterial and by synthetic polymer. The synthetic polymer is thermoformable synthetic polymer or thermoset synthetic polymer, or the combination of them. The thermoformable synthetic polymer chosen from the thermoformable synthetic polymer's with fluidity index between 0,1 to 100 g/10 minutes, chosen from thermoplastic resins with processing temperature between 100 and 350° C. The thermosets polymer chosen from elastomers such as Polyisoprene, natural rubber, polybutadiene, SBS, scone rubbers, nitrilic rubber, and chloroprene rubber. Preferably, the thermoset polymer chosen from silicone rubbers.

Preferably, the synthetic polymer is thermoformable synthetic polymer with fluidity index between 1 to 5000 minutes, chosen from thermoplastic resins with processing temperature between 100 and 350″C.

Thermoplastic resins are preferably chosen from polypropylene PP, polyethylene PE, polystyrene PS, polyesters, polyoxymethylenes, polyethylene-terephthalate, polybutylene-terephthalate, polymethylmethacitalates, polyethersulfones, polysulfones, polyester-ketones, polystyrene-copolymers, acrylonitrile-butadiene-styrene butadiene styrene, polyamides, nylon 6, nylon 6,6, polyvinyl chloride and/or combinations thereof combinations, polyolefins or polycycloolefins, such as: polyisobutylene, poly-1-butene, poly-4-methyl butene, poly-4-methyl-1-pentene, polyvinylcyclohexane, polyisoprene, polybutadiene, high density polyethylene HOPE, ultra-high molecular weight high density polyethylene HOPE, medium density polyethylene MOPE, low density polyethylene LOPE density LOPE, linear low density polyethylene LLDPE, linear ultra-low density polyethylene density linear polyethylene ULDPE, polypropylene/polyethylene PP/HOPE, blends of different types of polyethylene, e.g. HOPE/LOPE, polypropylene/polyisobutylene, among others. Thermoplastic resins may preferably also be blends and solutions thereof, homopolymers and/or copolymers, such as ethylene/propylene copolymers; propylene copolymers/1-butene, isobutylene/propylene copolymers, ethylene/1-butene copolymers; ethylene/hexene copolymers, ethylene/methylpentene copolymers, ethylene/heptene copolymers, ethylene/cyclolethin copolymers, propylene/butadiene copolymers, isobutylene/isoprene copolymers, ethylene/alkylacrylate copolymers, ethylene/vinyl acetate copolymers or their combinations, ethylene/propylene copolymer/propylene, LDPE/ethylene copolymer/EVA vinyl acetate, prevented stereo polymers, among others. And homopolymers and copolymers can comprise sindiosthetic, isostatic, astatic, or hemi-isostatic, structures.

NPE material can be produced by any known process that dispersion of solid substances occurs in a polymeric matrix, such as the fusion mixing method, in situ polymerization and fusion mixture, without limiting themselves to these. Preferably the Production Method of NPE is by fusion mixture, according to Example 1. For the formation of plastic products, the NPE material can be used as obtained or used in the form of masterbatches.

The NPE material used in the form obtained for the manufacture of plastic products contains concentrations of metal catalyst made of alkaline nanostructured nanomaterial between 1 ppm and 700,000 ppm, preferably in the range of 1 ppm to 10,000 ppm.

The NPE material used in the form of masterbatches for the manufacture of plastic products contains concentrations of metal catalyst made of alkaline nanostructured material in the range of 100,000 ppm to 900,000 ppm, preferably in the range of 100000 ppm to 750,000 ppm.

NPE material can have any format, such as, for example, but without limiting themselves to them, tile, plate, doll, cone, box, thin film, cup, etc. NPE material can be a transparent, translucent or opaque material. NPE material can be a colorless material or a colored material. The NPE material can be porous or solid, in this case, when solid, it is without permeability to gases. The NPE material can present the inner face and the external face with smooth texture, or present the inner face and the outer face with rough texture. The NPE material can present one of the faces, internal or external, smooth and the other face, internal or external, rough. The NPE material can present natural texture on the inner and outer faces, or present at least one of the faces, internal or external, with natural texture and the other face, internal or external, with rough or smooth texture. The NPE material presents variable thickness, defined according to its use, such as for example, for the use of NPE material as thin film, it, the NPE material, can have thickness on the angstrom or nanomeric scale, already for use in plate format, the NPE material can have thickness in the millimeter or micrometric scale; and so on.

The best configuration for NPE material, which provides you with the best speed and performance in GPHU production, as well as the configuration that best provides the release and dispersal of GPHU in the environment naturally by the surface of the photoactivated NPE material, is when it, the special configuration of the NPE material, comprises at least the following characteristics: opaque, with at least gas porosity and rough texture on at least one of the faces, preferably with rough multifocal texture, as explained below.

Knowing that a certain UV/Vis radiation can be transmitted completely by a transparent material, transmitted partially by a translucent material and that it is not transmitted through opaque materials, then preferably the NPE material is an opaque material, because then UV light used to light-activate the NPE material, is mostly absorbed by the catalyst and the polymer matrix of the NPE material. As the inventive method refers preferably to an open system, it is of interest that UV light is not transmitted to the environment. When the thickness of the NPE material is about 1 mm, more than 95% of the UVA light incident on the NPE material is absorbed and less than 1% is transmitted through it to the environment, totally losing energy less than 1 meter from the light source.

The NPE material is preferably porous at least in gases, because this feature allows the GPHU produced internally in the photoactivated NPE material to also be released naturally through the surface of it, the NPE material. The porosity at least the gases contributes to the releasing of GPHU to the environment.

The rough property preferably attributed to the NPE material because it provides for GPHU produced and released in various directions of the environment. In addition, roughness increases the surface contact area of the catalyst contained in the NPE material with the incident UV light for its activation, increasing the speed and yield of reactions.

The metallic catalyst made of alkaline nanostructured nanomaterial and dispersed in the polymer matrix of the NPE material, is photoactivated by UVA light energy with wavelength between 320 nm to 400 nm, or UVB with wavelength between 290 nm to 320 nm, or UVC with a length between 200 nm to 290 nm, or by the combination of them. The catalyst has band gap with energy in the UVA light energy range. Therefore, preferably the said catalyst is photoactivated by UVA light energy. A technician on the subject knows that for the activation of any catalyst it is necessary that the luminous energy is equal to or greater than its activation energy. The UVC light energy and UVB light energy are more energetic than UVA light energy and therefore the light energy UVC and the light energy UVB, can also be used to photoactivate the said catalyst.

However, when the metallic catalyst made of alkaline nanostructured nanomaterial is photoactivated with UVC light energy with a wavelength equal to or less than 254 nm, the GPHU immediately after being produced is ionized to a hydroxyl radical by photolysis, according to Equation 8, giving rise to the Ionized Ultrapure Hydrogen Peroxide Gas GPHUi.

H2O2+hv→2OH· Equation 8

Therefore, for the production and release of GPHU to the environment, the metal catalyst made of alkaline nanostructured nanomaterial should be photoactivated by UVA light energy and/or UVB light energy, and/or by UVC light energy with wavelength above 254 nm up to 290 nm. Preferably the metallic catalyst made of alkaline nanostructured nanomaterial is photoactivated by UVA light energy for production and release of GPHU to the environment. UVA and UVB light energies can be of natural/solar source or artificial source. Preferably, UVA and UVB light energies are by artificial source. The UVC light energy is only from an artificial source. The UVC light energy with wavelength equal to or less than 254 nm is used when interest is the production of the ionized form of GPHU, the GPHUi.

The ionized form of GPHU, the GPHUi, can also be obtained when photoactivated NPE material is used in a closed system. In this case, the GPHU produced will be oxidized by the action of catalytic plasma.

GPHUi, i.e. ionized GPHU in the form of hydroxyl radicals, is highly reactive and can oxidize most compounds, especially organic ones. The most powerful oxidants, relative to their standard potentials, in Volt, follow the following order: fluorine (3,0 V)>hydroxyl radical (2,8 V)>ozone (2,1 V)>hydrogen peroxide (1,77 V)>potassium permanganate (1,7 V)>chlorine dioxide (1,5 V)> and chlorine (1,4 V). The high oxidation power associated with its use does not generate waste, the ionized form of the GPHU, the GPHUi, is also considered an important ecological decontamination agent and can be used, for example, as environmental decontaminant of air, water and soil and as a sterilizer of medical/hospital equipment, among other applications, without limiting them.

When in the gaseous environment, GPHU can be inhaled and entered the bloodstream by diffusion in the pulmonary alveolus, acting as a therapeutic agent in bioxidative therapies, applied to humans and animals, including low catalysis animals such as dog and chicken. In a simplified analysis, in the bloodstream the GPHU is neutralized in 2H⁺ ions+O₂· (singleton oxygen), by blood plasma components, such as albumins, together with cellular cytoplasm components such as antioxidant enzymes: catalase, glutadione, GHS-PX peroxidase and thyrodoxins, activating a natural protection process called hormesis, defined as an adaptive response of cells and organisms to moderate, usually intermittent stress.

The hormesis process generates several systemic benefits to the body, acting on the immune system, the cytoplasm and cell nucleus and organs. The main effects on the body are: hyper oxygenation, important for systemic regulation of the body, as well as in the treatment of cancer and infections; alkalinity; more energy; angiogenesis, the effect is physiological and does not interfere with cancer, favoring cell invigoration, healing of open wounds, and vascular problems; decreases the excess oxidative stress; detox action, eliminates xenobiotics and heavy metals; vasodilation of the cerebral arteries; increased serotonin synthesis; increases antioxidants; improves the oxygen transport and vascular conditions such as atherosclerosis, thrombosis, etc.; decreases platelet agglomeration, preventing the formation of dots and thrombi; dissolution of atheroma plaques; greater vasodilation and elasticity of blood vessels, both peripheral and coronary; decreases the agglomeration of erythrocytes, known as the Rouleau effect; improves thyroid health and type II diabetes; avoids ferrotoxicity; improves adrenal health; in the liver causes physiological increase in insulin secretion, sensitivity of liver receptors and synthesis of glycogen; decreases excess ferritin; physiological stimulation of the adrenal gland, favoring the formation of testosterone and cortisol, in case it is low.

GPHU can also be used for food conservation purposes, such as vegetables, pasta and meat, without limiting themselves to them, acting on the biodisinfection of food surfaces, according to Example 2, and for the biodisinfection of air and surfaces in empty environments and in inhabited environments. Inhabited environment refers to an ecological or environmental area that is inhabited by a particular species of animal, plant or other organism. Empty environment refers to an ecological or environmental area that is not inhabited. GPHU has biocide action for many pathogenic microorganisms, including bacteria, viruses, fungi, spores; yeasts and fungi. The free energy variation of Gibbs DG° associated with the breakdown of the covalent bond between the two oxygen atoms in H₂O₂ is relatively low, about 213 kJ mol⁻¹, makes that when in contact with microorganisms, the GPHU is oxidized to hydroxyl radicals; and they, hydroxyl radicals, can damage nucleic acids; proteins and lipids of various types of microorganisms.

Given the above, it is clear that the photoactivated NPE material can be used for the production and natural release of GPHU in the gaseous and aqueous environment for uses, contemplating the manufacture of materials and products for therapeutic purposes, in the area of health and aesthetics; for the conservation of foods, such as vegetables; pasta and meat, without limiting themselves to them; and for the biofedify of air and surfaces of empty and inhabited environments; for the production of oxygenated water from GPHU; among other applications, without limiting themselves to these.

The NPE material can be also used for the manufacture of materials and products for plasma production purposes containing highly reactive species, mainly reactive species of oxygen ERO and hydroxyl radicals, when the catalytic cell containing photoactivated NPE material is in a closed system. The Plasma can be used as environmental decontaminant of air, water and soil and as a sterilizer of medical/hospital equipment, among other applications, without limiting itself to these applications.

The NPE material can be also used in other systems such as acting as a catalytic cell in electrocatalysis devices and solar panels for the conversion of solar energy into electric energy.

The inventive method brings a device for the synthesing and releasing of GPHU to the gaseous environment for use in bioxidative therapies via inhalation, applied to humans and animals, formed by NPE material containing metallic catalyst made of alkaline nanostructured nanomaterial, UVA light and power cord to connect UVA light to the electrical grid. This appliance also used for the biodisinfection of air and surfaces in empty environments or in inhabited environments. The device may also contain one or more sensors, such as humidity, temperature, hydrogen peroxide, and ozone, without limiting them. The appliance can used indoors or in a ventilated environment. Preferably, the device is used in a ventilated environment. Preferably, the appliance is used as an open system, that is, without enclosure.

### Examples

Example 1 refers to a form; without limiting it, of the production of NPE material, which includes a form of production; without limiting itself to it, of metallic catalyst made of an alkaline nanostructured material. The production of said catalyst is carried out by the hydrothermal treatment method: in a reactional environment 2 M to 7 M of metal oxide of nanoparticulate TiO₂, with average particle diameter of 30 nm, is added in an aqueous solution containing 5 M to 15 M of sodium hydroxide and inserted in an autoclave and heated between 100° C. to 300° C., in between 24 hours to 72 hours. Next the catalyst obtained is coated superficially with organic additive of the vegetable oil type, through physical mixture. Next the metal catalyst made of an alkaline nanostructured material, coated with organic additive, is mixed with polyethylene thermoformable synthetic polymer pellets. For the production of the NPE material, method used was the mixture by fusion, at temperature between 100° C. to 350° C. The equipment used for fusion mixing was thermomechanical equipment of the double screw extruder type. The Special Polymeric Nanomaterial NPE was molded as obtained, in the form of plates with thickness of 1 mm, with rough texture of the multifocal type on one side.

Example 2 refers to the analyses of the use of GPHU for the conservation of fruits and vegetables foods, without limiting itself to it. The analysis was carried out in cold chambers storage of fruits and vegetables of a supermarket chain. The camera has an area of 100 m². The methodology consisted of: in a chamber (1) of 100 m² NPE material plates photoactivated with UVA light were placed for a period of 15 minutes every 1 hour, and a sample of each fruits and vegetables, containing boxes of each type. The period of analysis varied for each type of fruits and vegetables. For example, for papaya the analyses lasted 60 days, for banana 70 days, and for tomato 40 days, In another chamber (2) the same types and quantities of the chamber samples (1) were placed, but the chamber (2) did not contain the photoactivated NPE material plates. The results showed that for the analyzed fruits and vegetables, the lifetime increased more than 80% when was in the cold chamber storage (1) containing the NPE material plates photoactivated with UVA light.

1. METHOD FOR SYNTHESIS AND RELEASING ULTRA-PURE HYDROGEN PEROXIDE GAS comprising: ultra-pure hydrogen peroxide gas, metal catalyst made of alkaline nanostructured nanomaterial, special polymeric nanocomposite material and ultraviolet light.

2. METHOD FOR SYNTHESIS AND RELEASING ULTRA-PURE HYDROGEN PEROXIDE GAS according to claim 1 wherein said ultra-pure hydrogen peroxide gas is naturally released to the environment by the surface of the special polymeric nanocomposite material photoactivated by ultraviolet light.

3. METHOD FOR SYNTHESIS AND RELEASING ULTRA-PURE HYDROGEN PEROXIDE GAS according to claim 1 wherein said ultra-pure hydrogen peroxide gas is naturally released to the environment by the surface of the special polymeric nanocomposite material photoactivated by ultraviolet light is the release speed rate being between 2,2×10¹¹ nm/s to 4,4×10¹¹ nm/s at room temperature.

4. METHOD FOR SYNTHESIS AND RELEASING ULTRA-PURE HYDROGEN PEROXIDE GAS according to claim 1, wherein said ultra-pure hydrogen peroxide gas is with average concentration in steady state between 0,001 parts per million to 5 parts per million in the environment.

5. METHOD FOR SYNTHESIS AND RELEASING ULTRA-PURE HYDROGEN PEROXIDE GAS according to claim 1 wherein said ultra-pure hydrogen peroxide gas is being substantially free of hygroscopic substances and substantially metal-free.

6. METHOD FOR SYNTHESIS AND RELEASING ULTRA-PURE HYDROGEN PEROXIDE GAS according to claim 1 wherein said special polymeric nanocomposite material is having crystalline structure formed by metal catalyst made of alkaline nanostructured nanomaterial and by synthetic polymer.

7. METHOD FOR SYNTHESIS AND RELEASING ULTRA-PURE HYDROGEN PEROXIDE GAS according to claim 1 wherein said metal catalyst made of alkaline nanostructured nanomaterial is able to present dimensionality 1 D, or 2D or 3D, or the combinations between them.

8. METHOD FOR SYNTHESIS AND RELEASING ULTRA-PURE HYDROGEN PEROXIDE GAS according to claim 1, wherein said metal catalyst made of alkaline nanostructured nanomaterial is preferably 2D dimensionality.

9. METHOD FOR SYNTHESIS AND RELEASING ULTRA-PURE HYDROGEN PEROXIDE GAS according to claim 1 wherein said metal catalyst made of alkaline nanostructured nanomaterial is having crystalline structure formed by metallic oxide and by alkalis in its structure.

10. METHOD FOR SYNTHESIS AND RELEASING ULTRA-PURE HYDROGEN PEROXIDE GAS according to claim 9 wherein said metallic oxide contained in the crystalline structure of the metal catalyst made of alkaline nanostructured nanomaterial is being chosen from TiO₂ anatase, or ZnO, or CuO, or WO₃, or Al₂O₃, or Nb₂O₅, or In₂O₃, or Fe₂O₃, or SnO₂, or SrTiO₃, or Nb₂O₅/MoO₃, or ZnS, or CdS, or be one or more combinations between them.

11. METHOD FOR SYNTHESIS AND RELEASING ULTRA-PURE HYDROGEN PEROXIDE GAS according to claim 9, wherein said metallic oxide contained in the crystalline structure of the metal catalyst made of alkaline nanostructured nanomaterial is being preferably chosen from TiO2 anatase.

12. METHOD FOR SYNTHESIS AND RELEASING ULTRA-PURE HYDROGEN PEROXIDE GAS according to claim 9, wherein said metallic oxide contained in the crystalline structure of the metal catalyst made of alkaline nanostructured nanomaterial is having 0D dimensionality with nanoparticle shape, with average diameter of the particles between 1 nanometer and 100 nanometers, preferably with average particle diameter between 1 nanometer and 30 nanometers.

13. METHOD FOR SYNTHESIS AND RELEASING ULTRA-PURE HYDROGEN PEROXIDE GAS according to claim 9, wherein said metallic oxide contained in the crystalline structure of the metal catalyst made of alkaline nanostructured nanomaterial is able to present a surface covered with metal co-catalyst chosen from Ag, Au, or Pt, or Al, or Ru, or Co, or Cr, or Cu, or Rh, or Nb, or Mg, or Si, or Sn, or Pd, or Ti, or Ir, or Zn, or Ge, or Mo, or Fe, Ni, or W, or Ta, or one or more combinations between them.

14. METHOD FOR SYNTHESIS AND RELEASING ULTRA-PURE HYDROGEN PEROXIDE GAS according to claim 9 wherein said alkalis contained in the crystalline structure of the metal catalyst made of alkaline nanostructured nanomaterial is being chosen from Li, or Na, or K, or Rb, or Cs, or Be, or Mg, or Ca, or Sr, or Ba, or Ra, or be one or more combinations between them.

15. METHOD FOR SYNTHESIS AND RELEASING ULTRA-PURE HYDROGEN PEROXIDE GAS according to claim 9 wherein said alkalis contained in the crystalline structure of the metal catalyst made of alkaline nanostructured nanomaterial is being preferably chosen from Li, Na, and Mg or be one or more combinations between them.

16. METHOD FOR SYNTHESIS AND RELEASING ULTRA-PURE HYDROGEN PEROXIDE GAS according to claim 1, wherein said metal catalyst made of alkaline nanostructured nanomaterial is being preferably chosen from sodium titanate with 2D dimensionality with nanowall format, or lithium titanate with 2D dimensionality with nanowalls format, or magnesium titanate with 2D dimensionality with nanowalls format, or be one or more combinations between them.

17. METHOD FOR SYNTHESIS AND RELEASING ULTRA-PURE HYDROGEN PEROXIDE GAS according to claim 1, wherein said metal catalyst made of alkaline nanostructured nanomaterial is san able to present surface covered with metal co-catalyst chosen from Ag, Au, or Pt, or Al, or Ru, or Co, or Cr, or Cu, or Rh, or Nb, or Mg, or Si, or Sn, or Pd, or Ti, or Ir, or Zn, or Ge, or Mo, or Fe, Ni, or W, or Ta, or one or more combinations between them.

18. METHOD FOR SYNTHESIS AND RELEASING ULTRA-PURE HYDROGEN PEROXIDE GAS according to claim 17 wherein said metal co-catalyst that may be covering the surface of the metal catalyst made of alcaline nanostructured nanomaterial is preferably chosen from Ag, or Au, or Al, or one or more combinations of them.

19. METHOD FOR SYNTHESIS AND RELEASING ULTRA-PURE HYDROGEN PEROXIDE GAS according to claim 1, wherein said metal catalyst made of alkaline nanostructured nanomaterial is presenting surface covered with organic additive.

20. METHOD FOR SYNTHESIS AND RELEASING ULTRA-PURE HYDROGEN PEROXIDE GAS according to claim 19 wherein said organic additive covering the surface of the metal catalyst made of alkaline nanostructured nanomaterial is able to be chosen from vegetable oil, or mineral oil, or fatty material, or CTAB cetyltrimethylammonium bromide, or grafted copolymer (PP-g-MA), or be one or more combinations between them.

21. METHOD FOR SYNTHESIS AND RELEASING ULTRA-PURE HYDROGEN PEROXIDE GAS according to claim 19 wherein said organic additive covering the surface of the metal catalyst made of alkaline nanostructured nanomaterial is being preferably vegetable oil.

22. METHOD FOR SYNTHESIS AND RELEASING ULTRA-PURE HYDROGEN PEROXIDE GAS according to claim 1 wherein said metal catalyst made of alkaline nanostructured nanomaterial is visually well distributed and with no agglomeration in the special polymeric nanocomposite material.

23. METHOD FOR SYNTHESIS AND RELEASING ULTRA-PURE HYDROGEN PEROXIDE GAS according to claim 6 wherein said synthetic polymer present in the crystalline structure of the special polymeric nanocomposite material is thermoformable synthetic polymer or thermosetting synthetic polymer, or being the combination between them.

24. METHOD FOR SYNTHESIS AND RELEASING ULTRA-PURE HYDROGEN PEROXIDE GAS according to claim 6 wherein said synthetic polymer present in the crystalline structure of the special polymeric nanocomposite material is preferably thermoformable polymer with fluidity index between 1 to 100 g/10 minutes, preferably with fluidity index between 1 to 50 g/10 minutes.

25. METHOD FOR SYNTHESIS AND RELEASING ULTRA-PURE HYDROGEN PEROXIDE GAS according to claim 23 wherein said thermoformable synthetic polymer present in the crystalline structure of the special
polymeric nanocomposite material is preferably chosen from thermoplastic resins with processing temperature between 100° C. and 350° C.

26. METHOD FOR SYNTHESIS AND RELEASING ULTRA-PURE HYDROGEN PEROXIDE GAS according to claim 23 wherein said thermosetting synthetic polymer when present in the crystalline structure of the special polymeric nanocomposite material is chosen from Polyisoprene, or natural rubber, or polybutadiene, or SBS, silicone rubber, or nihilistic rubber, or chloroprene rubber, or be one or more combinations between them.

27. METHOD FOR SYNTHESIS AND RELEASING ULTRA-PURE HYDROGEN PEROXIDE GAS according to claim 23 wherein said thermosetting synthetic polymer when present in the crystalline structure of the special polymeric nanocomposite material is being preferably chosen from silicone rubbers.

28. METHOD FOR SYNTHESIS AND RELEASING ULTRA-PURE HYDROGEN PEROXIDE GAS according to claim 1, wherein said special polymeric nanocomposite material is containing concentration of metal catalyst made of alkaline nanostructured nanomaterial in the range of 1 ppm to 700,000 ppm, preferably in the range of 1 ppm to 10,000 ppm, when it, the special polymeric nanocomposite material is used as obtained.

29. METHOD FOR SYNTHESIS AND RELEASING ULTRA-PURE HYDROGEN PEROXIDE GAS according to claim 1 wherein said special polymeric nanocomposite material is containing concentration of metal catalyst made of alkaline nanostructured nanomaterial in the range of 100,000 ppm a 900,000 ppm, preferably in the range of 100,000 ppm a 750,000 ppm, when it, the special polymeric nanocomposite material is used in the form of masterbatches.

30. METHOD FOR SYNTHESIS AND RELEASING ULTRA-PURE HYDROGEN PEROXIDE GAS according to claim 1, wherein said special polymeric nanocomposite material is an opaque material, with porosity at least gases and with rough texture on at least one of the faces, preferably with rough texture of the multifocal type on at least one face.

31. METHOD FOR SYNTHESIS AND RELEASING ULTRA-PURE HYDROGEN PEROXIDE GAS according to claim 1, wherein said metal catalyst made of alkaline nanostructured nanomaterial is being photoactivated by ultraviolet energy with UVA wavelength between 320 nm to 400 nm, or with UVB wavelength between 290 nm to 320 nm, or with a larger UVC wavelength than 254 nm and less than 290 nm, or by one or more combinations between them.

32. METHOD FOR SYNTHESIS AND RELEASING ULTRA-PURE HYDROGEN PEROXIDE GAS according to claim 31 wherein said ultraviolet energy used to photoactivated the metal catalyst made of alkaline nanostructured nanomaterial is being preferably ultraviolet energy with UVA wavelength between 320 nm to 400 nm.

33. METHOD FOR SYNTHESIS AND RELEASING ULTRA-PURE HYDROGEN PEROXIDE GAS according to claim 1 wherein said ultra-pure hydrogen peroxide gas produces oxygenated water when released into the aqueous environment.

34. ULTRA-PURE HYDROGEN PEROXIDE GAS obtained by the method defined in claim 1, wherein said Ultrapure Hydrogen Peroxide Gas is substantially free of hygroscopic substances and substantially metal-free.

35. ULTRA-PURE HYDROGEN PEROXIDE GAS according to claim 1 wherein said ultra-pure hydrogen peroxide gas is presenting average steady state concentration between 0,001 parts per million to 5 parts per million in the environment.

36. SPECIAL POLYMERIC NANOCOMPOSITE MATERIAL obtained by the method defined in claim 1, wherein said special polymeric nanocomposite is producing and releasing the ultra-pure hydrogen peroxide gas to the environment.

37. SPECIAL POLYMERIC NANOCOMPOSITE MATERIAL, as claimed 36 wherein said Special Polymeric Nanocomposite is being photoactivated by ultraviolet light.

38. APPARATUS according to claim 1, wherein said apparatus synthesize and release ultra-pure hydrogen peroxide gas for use and is formed by special polymeric nanocomposite material containing metal catalyst made of alkaline nanostructured nanomaterial, UVA light and power cord to connect UVA light to the electrical network.
